(19) United States
(12) Patent Application Publication
(10) Pub. No.: US 2020/0188154 A1
(43) Pub. Date: Jun. 18, 2020

(54) Knee Brace Massager Apparatus

(71) Applicant: Raymond Hsu, Shenzhen City (CN)

(72) Inventor: Raymond Hsu, Shenzhen City (CN)

(21) Appl. No.: 16/296,059

(22) Filed: Mar. 7, 2019

Related U.S. Application Data
(63) Continuation-in-part of application No. 29/673,390, filed on Dec. 14, 2018.

(51) Int. Cl. A61F 5/01; A61F 7/00; A61H 7/00; A61N 1/04; A61N 1/32; A61N 5/06

(52) U.S. Cl. CPC A61F 5/0109; A61F 7/007; A61H 7/004; A61N 1/0456; A61N 1/322; A61F 2007/0225; A61H 2201/1671; A61H 2205/102; A61N 2005/0654; A61N 2005/0645; A61N 5/0613

(57) ABSTRACT

An apparatus for knee massage comprises a hollow L-shape structure that convexes to fit a user's knee. The L-shape structure further comprises an open top, an open back, and a hollow cavity to allow the user's knee rests within. The hollow cavity further comprises an array of massage devices disposed about the inner corner of the L-shape structure. The apparatus further comprises a CPU and PCB. The CPU processes the user's commands and controls the apparatus' massage functionality. The PCB further comprises a wireless communication component that processes request and response communication between the user and the apparatus. The apparatus further comprises a rechargeable lithium battery component. The PCB further comprises a charger component. The CPU controls the charger component and charging activities.

### INCORPORATION BY REFERENCE

This continuation in part application claims the benefit of priority to the filing date of U.S. non provisional design patent application No. 29/673,390, entitled “Knee Brace Massager”, which was filed on Dec. 14, 2018, and which is incorporated herein by reference in its entirety.

### FIELD OF INVENTION

The current invention relates to personal and portable massage machines, and particularly knee massage machines.

### BACKGROUND OF THE INVENTION

As a higher quality of life, improvements in medicine, and a better diet have increased the average life span of people, new obstacles have arisen as more of the population reaches a higher age than previously encountered by humans, and additional strain is put on the body itself. In particular, as people age, their joints are affected by thinning of the cartilage and in connective tissue and, thus, stiffness or aches and pains are expected to be encountered.

Additionally, some of the populace has a life-style that puts additional pressures on the body, such as obesity or an intensively active lifestyle, like distance running, that will likely result in joint pain and injury.

Besides Western medicine that attempts to eliminate the root cause of the diseases, therapeutic medicine originating from the East is now increasingly being viewed as having positive therapeutic benefits, especially, where they alleviate chronic sore or pain and improve quality of life, some of which are now even measurable in medical studies. To some extent, some now view the Western remedies of surgery or drugs as extreme, unnecessary, or undesirable and wish to explore less invasive remedies in an attempt to alleviate pain.

It is estimated that the overall prevalence of knee pain for men of all ages is between 15 and 20%. A study in the US showed that approximately 18% of men aged 60 years and older reported knee pain and the incidence increases steadily with age. The incidence of knee pain has been found to be slightly more common in women compared to men. The overall prevalence of knee pain in women is approximately 20%. A study in the US showed that approximately 23% of women aged 60 years and older reported knee pain.

To meet the increasing demand of the aging population, many massage therapies to alleviate sores or pains have been invented and introduced. One of the therapies is Transcutaneous Electrical Nerve Stimulation (TENS) therapy which involves the use of low-voltage electric currents to treat pain. Electrodes or mediums for electricity to travel to the body, placed on the body at the site of pain deliver electricity that travels through the nerve fibers. The electricity from the electrodes stimulates nerves in the affected area and sends signals to the brain that “scramble” normal pain perception. The electric currents block the pain receptors from being sent from the nerves to the brain.

Today, typically a patient receiving TENS therapy will receive a small kit, which includes a low voltage battery since a TENS device draws small amount of electricity to operate. The unit is usually connected to the skin using two or more electrodes. A typical battery-operated TENS unit is able to modulate pulse width, frequency and intensity. Generally, TENS is applied at high frequency (>50 Hz) with an intensity below motor contraction (sensory intensity) or low frequency (<10 Hz) with an intensity that produces motor contraction.

Another pain remedy is “Teding Diancibo Pu” therapy, or, as it is more commonly known, TDP treatment. The TDP device comprises an array of lamps that emit the far infrared light or mid-IR wavelengths on the treatment site of the body. The lights the lamps emit can loosen the fibrous tissues connecting the muscles and organs. Blood can thus circulate more freely to deliver fresh oxygen, nutrients, and healing enzymes to the body's cells, and also work to carry away toxins, dead cells, debris, and other waste. In this way, the device stimulates the body to heal itself in a process similar to the healing energy that radiates from the hands of a massage therapist. The device is also believed to work according to the basis of Traditional Chinese Medicine or TCM by promoting the free movement of “qi,” “chi” or life energy in the body.

A typical TDP lamp resembles a traditional incandescent lamp and similarly can be mounted on a movable base with a vertical pole, spring arm, and head so it can be adjusted in all manner of positions. It also includes a timer, either mechanical or digital. The wire cage head of the lamp is comprised of a heat reflector, insulator, mineral plate, and heating plate activated with electricity. The key component of the TDP Lamp is the mineral plate that is heated to 870° F. or 466° C. This device can emit far infrared radiation that ranges from 2 to 50 micrometers. The TDP Mineral Lamp is a relatively new therapeutic device that treats ailments by emitting far infrared light. This method is increasingly being used by physical therapists, chiropractors, acupuncturists, and other healthcare professionals to help their patients with chronic illnesses.

Although advanced and preferred, one shortcoming of the current state of the TDP lamp is the amount of physical space the device occupies. Another limitation is that a masseuse attempting to apply massage will have difficulty in massaging the skin surface as they themselves will be blocking the TDP lamp with their own body or be faced with constantly readjusting the lamp position. Fortunately, product design and production techniques have been improved to a point that a massager device can incorporate multiple functions while still being lightweight, compact, and portable.

As such, advances in manufacturing and increased efficiency have made it now possible in terms of space, volume, and cost, and now feasible and desirable to create a therapeutic unit that targets a particular joint area, incorporating massage and heating features, with the addition of a TDP lamp array. Such a device will allow the user to receive consistent and continuous massage and heat treatment, while simultaneously receiving the benefits of the TDP lamp treatment. Thus, it would seem desirable to have a cost-effective device that treats certain chronic knee pain at home with the same procedures that are traditionally only available at doctor or chiropractic office.

The TDP lamp itself works on the belief that the far infrared light or mid-IR wavelengths it emits can loosen the fibrous tissues connecting the muscles and organs. Blood can thus circulate more freely to deliver fresh oxygen, nutrients, and healing enzymes to the body's cells, and also work to carry away toxins, dead cells, debris, and other waste. In this way, the device stimulates the body to heal itself in a process similar to the healing energy that radiates from the hands of a massage therapist. The device is also believed to work according to the basis of Traditional Chinese Medicine or TCM by promoting the free movement of “qi,” “chi” or life energy in the body.

A typical TDP Lamp resembles a traditional incandescent lamp and similarly can be mounted on a movable base with a vertical pole, spring arm, and head so it can be adjusted in all manner of positions. It also includes a timer, either mechanical or digital. The wire cage head of the lamp is comprised of a heat reflector, insulator, mineral plate, and heating plate activated with electricity. The key component of the TDP Lamp is the mineral plate that is heated to 870° F. or 466° C. This device can emit far infrared radiation that ranges from 2 to 50 micrometers.

### OBJECTIVE OF THE INVENTION

It is the object of this invention to create a compact, portable, therapeutic massager device intended to target knee joint and soft tissue pain and to promote healing and easing of aches and pain.

It is the object of this invention to include a mechanical massage component to simulate traditional hand-masseuse massage.

It is further the object of this invention to incorporate a TDP lamp which targets the knee joint and soft tissue areas.

It is further the object of this invention to operate wirelessly, thus maximizing portability and convenience.

It is an object of this invention to deliver to the public a consumer grade, self-contained, easy to use therapeutic device that allows the user to self-administer an array of therapeutic functions to their own knees.

It is an object of this invention to allow for the home-user to safely self-administer “Teding Diancibo Pu” therapy, or, as it is more commonly known, TDP treatment or TDP mineral lamp treatment.

It is an object of this invention to include a heated massager that also compresses the knee joint to help reduce swelling, stiffness, and pain caused by arthritis, injuries, and other conditions, much in the same way traditional hand massage would.

It is an object of this invention to include Additional non-invasive therapeutic functions such as vibration, electric-stimulation, magnetic wave, and cooling functions.

It is an object of this invention to provide a cordless and portable massager device, that functions by way of a rechargeable lithium battery. As improvements in the consumer electronic device market have improved, more and more corded devices are disappearing from homes. Further, by enabling the device to be wireless, this will enable a person to travel with the device unlike a traditional TDP lamp. As such, it will now be possible for the afflicted to treat their aches and injuries at the office, gym, while traveling, and otherwise that were previously not possible or overly burdensome.

It is an object of this invention to allow a user from the comfort of their own home, office, or otherwise, with minimal equipment, to self-administer to their own knees the mechanical replication of traditional pressure hand massage, hot compress, and red light irradiation, specifically TDP electromagnetic waves, in order to heal and relieve knee pain.

### SUMMARY OF THE INVENTION

In one aspect of the invention, an apparatus for knee massage is disclosed comprises a hollow L-shape convex structure to fit a user's knee; wherein the L-shape structure comprises an open top, an open back, and a hollow cavity to allow the user's knee rests within; wherein the hollow cavity further comprises an array of massage devices disposed about the inner corner of the L-shape structure; wherein the apparatus further comprises a CPU and PCB; wherein the CPU processes the user's commands and controls the apparatus' massage functionality; wherein the PCB further comprises a wireless communication component; wherein the wireless communication component processes request and response communication between the user and the apparatus; wherein the apparatus further comprises a rechargeable lithium battery component; wherein the PCB further comprises a charger component; wherein the CPU controls the charger component and charging activities. In one embodiment, the array of massage devices comprises TENS electrodes. In one embodiment, the array of massage devices comprises TDP lamp mineral plates. In one embodiment, the array of massage devices comprises mechanical massage heads. In one embodiment, the mechanical massage heads move circularly. In one embodiment, the array of massage devices comprises RF heat applicators. In one embodiment, the PCB further comprises a RF energy source component capable of directing an RF power signal to the RF heat applicators.

In another aspect of the invention, a method of knee massage comprises providing a hollow L-shape convex structure fitting an user's knee is disclosed; wherein the L-shape structure comprises an open top, an open back, and a hollow cavity to allow the user's knee rests within; providing an array of massage devices disposed about the inner corner of the L-shape structure; providing a CPU and PCB; enabling the CPU to process the user's commands and to control the array of massage devices; providing a wireless communication component; enabling the wireless communication component to process request and response communication between the user and the CPU; providing a rechargeable lithium battery component; providing a charger component; enabling the CPU to control the charger component and charging activities.

### BRIEF DESCRIPTION OF THE DRAWINGS

These and other features and advantages of the invention will not be described with reference to the drawings of certain preferred embodiments, which are intended to illustrate and not to limit the invention, and in which:

FIG. 1 illustrates, from various orthogonal angles, an embodiment of this invention that comprises a 3-sided hollow structure.

FIG. 2 illustrates another embodiment of the invention that comprises a charging mechanism.

FIG. 3 illustrates various embodiments of the current invention with additional therapeutic capabilities.

FIG. 4 illustrates various mounting and/or attachment mechanisms that are used in concert with an embodiment of the current invention.

FIG. 5 illustrates another embodiment of this invention that comprises wireless communication, such as Bluetooth or Wi-Fi, and complex functionality.

### DETAILED DESCRIPTION OF THE DRAWINGS

The invention will be described in the context of a preferred embodiment unless otherwise indicated.

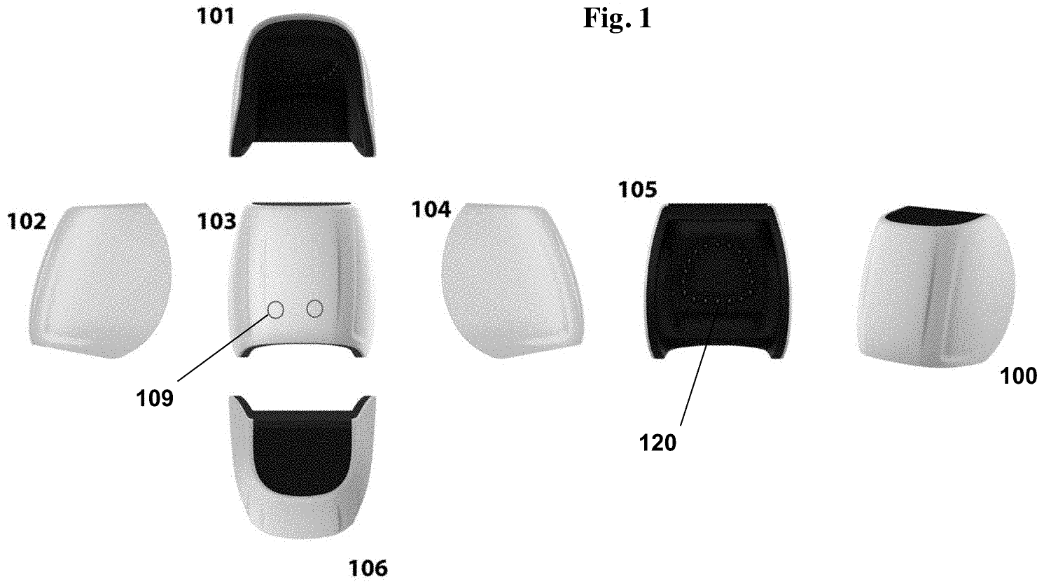

FIG. 1 illustrates the various orthographic views of an embodiment 100 of the current invention. 101 is the top view of the embodiment, 102 left thereof, 103 front thereof, 104 right thereof, 106 bottom thereof, and 105 the back thereof. 107 is the perspective view of the embodiment. The top 101 and back 105 are open to allow the user to rest his/her knee on the bottom inside the hollow cavity of the embodiment. Thus, the knee is completely surrounded by mechanical massage heads, heat points or electromagnetic points.

The embodiment 100 Further comprises status lights 109 which communicate battery charge levels or other functions, and venting holes 111 to accommodate the internal vacuum component. It is appreciated that the number of status lights 109 depends on the functions and/or therapies the device provides.

FIG. 1 further suggests various materials used in the construction of the embodiment. In an effort to keep production costs low, and, thus, the retail price low, a majority of the surfaces of the embodiment are to be of molded injected plastic, or a lightweight, durable, non-corrosive metal. It is preferable that the device remain relatively lightweight as it is meant to be held comfortably in the hand of consumers of all ages, for various lengths of time. It is preferable that the material whether plastic, metal, or composite, be non-corrosive as it is likely that the device will be stored in a person's bathroom and be exposed to water and humidity.

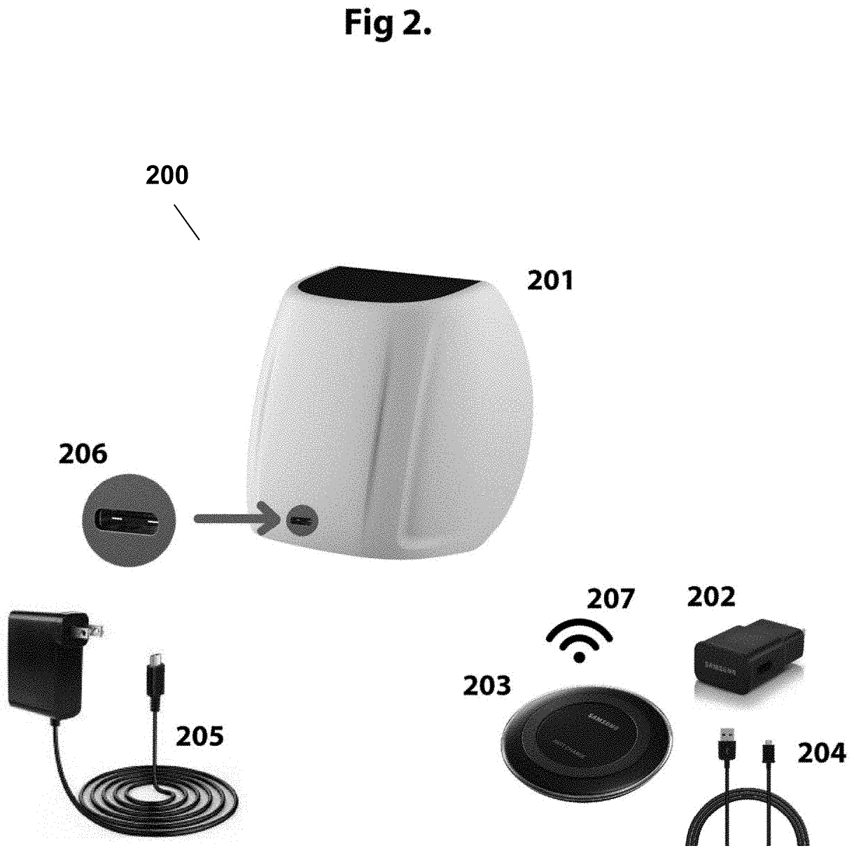

In this preferred embodiment, in an effort to maintain a minimal and modern aesthetic, there are no outlets to support a direct charging of the internal lithium battery. Instead, as will be discussed in FIG. 2, charging of the battery is achieved by way of wireless charging capability. It is nevertheless contemplated that in an alternative embodiment, there may be the addition of a USB-B or USB-C or other similar universal charging port such that a device user, at the sacrifice of some aesthetic, might recharge the battery of the device directly by cord connection. This may be preferable in instances of extended travel where a device user foresees that they will require recharging but does not want to be burdened with the addition of the wireless base and wants to instead use an external cable or charging pack for many different devices.

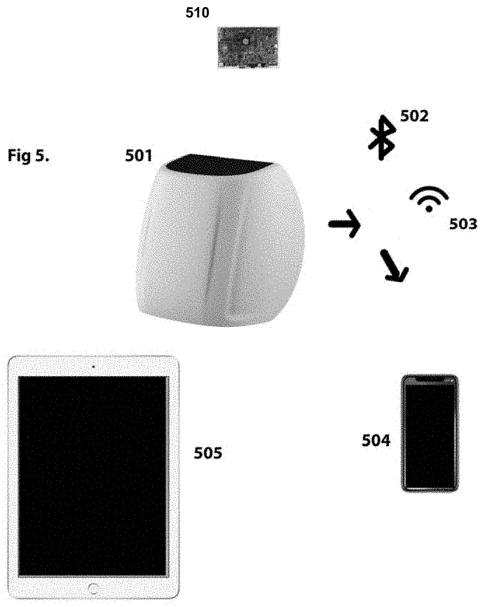

Control of the device function, like charging, will be achieved wirelessly, either by a nonspecific RF or other similar small remotes. enabling Bluetooth or Wi-Fi connectivity are contemplated and described in FIG. 5 such that the user's own cellphone may operate as the device controller.

A wireless massager device will enable a person to travel with it unlike a traditional TDP lamp massage equipment. As such, it will now be possible for users to treat their aches and injuries at the office, gym, while traveling, and otherwise that were previously not possible or overly burdensome.

The device itself may either be secured with the user holding the device in place, or in another embodiment, the device can be easily secured with adjustable straps.

As 105 view shows, all therapeutic functionality is contained in the inner hollow cavity of the device where the knee would be placed. In one embodiment, the “micro” TDP lamps 120 are disposed in an array surrounding the user's knee when it is placed inside the device for a treatment session.

A TDP lamp comprises a mineral plate made of iron that is about 0.5 to 1.5 millimeters in thickness. The plate serves as the base for the 33 trace elements in unique mineral forms that are also added in a layer. They are aluminum, antimony, arsenic, barium, boron, cadmium, calcium, carbon, chromium, cobalt, copper, iodine, magnesium, manganese, molybdenum, nickel, phosphorus, selenium, silicon, sodium, sulfur, tin, titanium, zinc, and zirconium along with bismuth, germanium, lithium, potassium, rubidium, strontium, and vanadium. The iron content of the plate helps the heat spread uniformly across the panel.

When the mineral plate in the TDP Lamp is heated to a high temperature, the minerals in it are ionized. The heat, radiation, and electromagnetic waves of the lamp carry these ions into the human body by penetrating up to 3½ inches below the surface of the skin and into the muscles. In their ionic state, the minerals can stimulate the healing and metabolic processes of the user's body.

In addition to the TDP lamp array, in the inner cavity of the device where the knee will be placed the device further comprises a massage mechanism. The massage mechanism can be mechanically driven in one embodiment and be a series of gear driven rotors which manipulate massage heads against the surface of the knee. Alternatively, or in addition to, air pressure may be utilized to compress and massage; the air bags will inflate and deflate to apply different levels of pressure on either side of the kneecap. This compression technique, employed by professional massage therapists, boosts circulation and increases body temperature to stimulate healing and prevent fluid collection causing inflammation.

FIG. 2 illustrates an embodiment 200 of the invention that comprises a charging mechanism. In this embodiment, the wireless charging 207 is achieved by way of a separate cradle 203 that is plugged into a traditional electrical outlet via cord 204 and male outlet adapter 202. The cradle 203 contains the necessary electronic components such that it can wirelessly charge the main units 201 internal lithium battery when the two components 201 and 203 are brought within close proximity of each other. In this embodiment, the main unit 201 can be lifted away with minimal effort since it is not attached to any charger cord.

It is appreciated that direct charging of the device can be enabled via the inclusion of a USB-C or other similar type charging port located on the device. In this embodiment the port 206 is located on the front of the device, but there may be cause or advantage to relocate this port to one of the other sides in other embodiments.

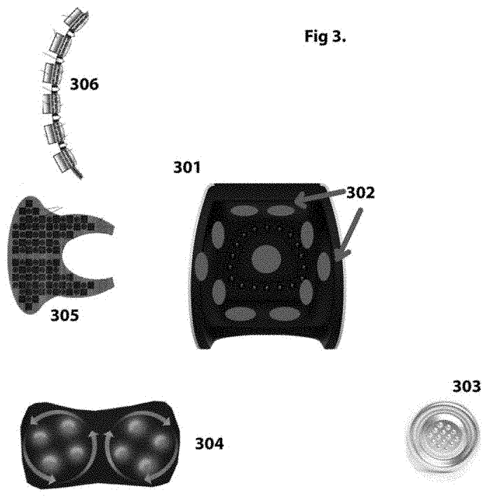

FIG. 3 illustrates another embodiment of the current invention that comprises additional therapeutic functions. This embodiment comprises a hollow back 301 that allows the user's knee to be placed. This embodiment further comprises an array of massage small devices 302. It is appreciated that massage devices 302 can be TDP mineral plates, TENS electrodes, or mechanical massage heads, or other massage therapy devices, and their arrangement can be calibrated for a certain treatment.

It is also appreciated that an increase the number of massage devices 302 contained within the device will provide a more rigorous massage and/or more variety of massage therapies.

It is contemplated that, in another embodiment, some of these massage devices 302 can be substituted with the addition of new therapies and functions.

In one embodiment, the massage devices 302 comprise metal massage heads 303. These Metal massage heads are conductive and can used to effectively transfer and treat areas of tissue with either cool temperatures or heat. In this embodiment, the metal massage head 303 would rests atop of a radiator and then electronically connected with a semiconductor refrigeration chip, in which a temperature sensor is inset in the semiconductor refrigeration chip, and the temperature sensor and semiconductor refrigeration chip are connected to the PCB main board. Alternatively, or concurrently, the metal massage head 303 can be connected to a heating element which would be similarly connected and controlled by the PCB main board. Unlike traditional hand massage, these computer controlled devices can direct precise heat and pressure as the user desires, repeatedly, and for as long as needed, any time of the day.

In an alternative embodiment, massage devices 302 comprise massage and heat head 304 that comprises a heat source and multiple mechanical micro massage heads. The heat head 304 applies simultaneously a lateral pressure to the tissue area to massage, while rotating the massage points on larger rotation points contained within, and maintains a low constant heat on the treatment site. It is anticipated that many consumers will desire different settings and configurations depending on their own needs.

Furthermore, this embodiment can further comprises TENS electrodes 305 and 306. These TENS electrodes are disposed around the hollow cavity of this embodiment in the array of massage devices 302. Thus, TENS therapy is easily incorporated in this embodiment as an additional and/or alternative therapy.

Transcutaneous electrical nerve stimulation (TENS) therapy involves the use of low-voltage electric currents to treat pain. Electrodes or mediums for electricity to travel to the body, placed on the body at the site of pain deliver electricity that travels through the nerve fibers. The electricity from the electrodes stimulates nerves in the affected area and sends signals to the brain that “scramble” normal pain perception. The electric currents block the pain receptors from being sent from the nerves to the brain.

Today, typically a patient receiving TENS therapy will receive a small kit, which includes a low voltage battery. The unit is usually connected to the skin using two or more electrodes. A typical battery-operated TENS unit is able to modulate pulse width, frequency and intensity. Generally, TENS is applied at high frequency (>50 Hz) with an intensity below motor contraction (sensory intensity) or low frequency (<10 Hz) with an intensity that produces motor contraction.

Since a TENS device's battery draw is small, it is easy to incorporate an additional TENS therapy function in an embodiment of the current invention without issue. Furthermore, as electrode points are small, a relatively complex grid 305 can be disposed within the embodiment. In addition, Because electrode arrays 306 are so flexible, they can be disposed following the curvature of the hollow cavity of the embodiment and knee. Depending on price point and user needs, a wide variety of functionalities can be incorporated in addition to merely massage and TDP therapies.

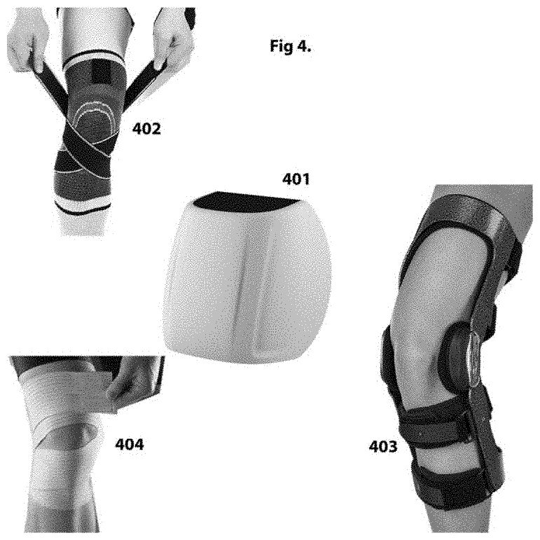

FIG. 4 illustrates different embodiments of the current invention with various mounting mechanisms, such as a strap or a Velcro adhesion.

In one embodiment 403, the massager device is mounted on a user's knee with an osteoarthritic knee brace having flexible upper and lower arm members rotatably connected to each other by a rotary hinge assembly that is often employed when significant injury has occurred. As it is likely that a person that is using this device has a weakened knee, there exists the possibility that they wish to use the device in conjunction with, or mounted over traditional physical supports.

In another embodiment 402, a slip on, nylon and elastic “slip-on” support mounting mechanism is used. This mounting mechanism is used more commonly by athletes or chronic pain sufferers.

Finally, in another embodiment 404, a traditional bandage wrap or ACE type bandage is used to mount a massage device of the current invention. Either 402, 403, or 404 mounting mechanisms can be used to mount the massage device 401 of the current invention over the knee and a user can utilize the massage functions.

Further, it is appreciated that additional mechanical or otherwise locking and/or attachment system can be used for a device user who has limited mobility and a need to keep the massage device in place for extended periods of time. However, whatever the mounting mechanism is employed, it is preferred the massage assembly exposes the knee so that the TDP therapy function will be most effective.

FIG. 5 illustrates an embodiment of this invention that comprises a wireless communication functionality, such as Bluetooth, NFC, IR, etc.

In this embodiment, massage device 501 is comprises electronic components, such as Bluetooth, Wifi, etc., that can communicate with a smartphone 504, or a tablet 505. This embodiment further comprises a CPU and PCB 510 that controls massage functions of the embodiment and its communication. The micro computer 510 allows for complex programming functionality and communication requests. Thus, it allows the device 501 to pair and communicate with smart mobile devices 504 or 505 via Bluetooth 502, wireless 503, or other direct protocol.

As disclosed above, embodiments of this invention can be rather complex in terms of the therapies it can provide. As the device becomes complex with more massage functions, a smart controller and software are required. As such, with smart devices 504 505, a user can set up pick massage functions, custom profiles, favorites, and so on. It is appreciated and anticipated that the massage device 501 can be shared among multiple users or home, or in a setting with even more volume, for instance in a sports injury clinic, or retirement home.

It is appreciated that with smart controller and software disclosed above, a user can have access to timer functions and tracking functions to keep track of therapy regimens, as well as, customizable settings that allow for a wide variety of functions, with many settings such as intensity of massage intensity of TDP lamps.

1. An apparatus for knee massage comprises a hollow L-shape convex structure to fit a user's knee; wherein said L-shape structure comprises an open top, an open back, and a hollow cavity to allow said user's knee rests within; wherein said hollow cavity further comprises an array of massage devices disposed about the inner corner of said L-shape structure; wherein said apparatus further comprises a CPU and PCB; wherein said CPU processes said user's commands and controls said apparatus' massage functionality;
wherein said PCB further comprises a wireless communication component; wherein said wireless communication component processes request and response communication between said user and said apparatus; wherein said apparatus further comprises a rechargeable lithium battery component; wherein said PCB further comprises a charger component; wherein said CPU controls said charger component and charging activities.

2. The apparatus of claim 1, wherein said array of massage devices comprises TENS electrodes.

3. The apparatus of claim 1, wherein said array of massage devices comprises TDP lamp mineral plates.

4. The apparatus of claim 1, wherein said array of massage devices comprises mechanical massage heads.

5. The apparatus of claim 4, wherein said mechanical massage heads move circularly.

6. The apparatus of claim 1, wherein said array of massage devices comprises RF heat applicators.

7. The apparatus of claim 6, wherein said PCB further comprises a RF energy source component capable of directing an RF power signal to said RF heat applicators.

8. A method of knee massage comprises
providing a hollow L-shape convex structure fitting an user's knee;
wherein said L-shape structure comprises an open top, an open back, and a hollow cavity to allow said user's knee rests within;
providing an array of massage devices disposed about the inner corner of said L-shape structure;
providing a CPU and PCB;
enabling said CPU to process said user's commands and to control said array of massage devices;
providing a wireless communication component;
enabling said wireless communication component to process request and response communication between said user and said CPU;
providing a rechargeable lithium battery component;
providing a charger component;
enabling said CPU to control said charger component and charging activities.
